(19) United States
(12) Patent Application Publication
(10) Pub. No.: US 2012/0330558 A1
(43) Pub. Date: Dec. 27, 2012

(54) IDENTIFICATION OF BIOMARKERS

(75) Inventors: Christian Baumgartner, Hall in Tirol (AT); Michael Netzer, Imst (AT); Bernhard Pfeifer, Schwaz (AT)

(73) Assignees: Medizinische Informatik und Technik), Hall in Tirol (AT); UMIT (Private Universitat fur Gesundheitswissenschaften,

(21) Appl. No.: 13/504,384

(22) PCT Filed: Oct. 26, 2010
(86) PCT No.: PCT/EP2010/066180

§ 371 Date: Sep. 13, 2012

Related U.S. Application Data
(60) Provisional application No. 61/255,769, filed on Oct. 28, 2009.

(51) Int. Cl. A61B 5/00; A61B 5/02; G01N 33/493; G06F 19/00; G01N 33/49

(52) U.S. Cl. 702/19

(57) ABSTRACT

The present invention relates to a method comprising the steps of obtaining a sample from the subject, measuring a plurality of biomolecules in the sample, identifying the measured biomolecules in the sample, characterized in that the method further comprises the steps of estimating a discriminatory ability of each measured biomolecule by using a paired test hypothesis, and integrating the estimated discriminatory abilities of the biomolecules into a kinetic analysis. More particularly, the present invention enables the use of such a method and a method for monitoring progress or treatment of a disease such as cardiovascular diseases. Such identification methods comprising the steps of obtaining a sample from the subject, measuring a plurality of biomolecules in the sample and identifying the measured biomolecules can particularly be used for monitoring progress or treatment of a disease.

The present invention relates to a method for identification of biomarkers in a subject according to the preamble of independent claim 1 and more particularly to a use of such a method and a method for monitoring progress or treatment of a disease such as cardiovascular diseases. Such identification methods comprising the steps of obtaining a sample from the subject, measuring a plurality of biomolecules in the sample and identifying the measured biomolecules can particularly be used for monitoring progress or treatment of a disease.

The search for new biomarkers in complex biological mixtures such as blood, urine or tissue have attracted attention to future-oriented clinical applications in the diagnosis, prognosis and treatment of cardiovascular disease (Ackermann et al., 2006, Kell, 2007, Howie-Esquivel et al., 2008, Gerszten et al., 2008, Lewis et al., 2008a) due to the rapid progress in mass spectrometry (MS) and in the development of related bioinformatics methods in recent years. For instance, biomarkers have a substantial impact on the care of patients with cardiovascular disease. For example, troponin is an accepted diagnostic marker for myocardial infarction and B-type naturetic peptide aids in diagnosis prognostication in myocardial infarction and heart failure (Sabatine et al., 2005; Collinson et al., 2007; Maisel et al., 2008).

However, there are not sensitive and specific early biomarkers of myocardial injury and therefore the complementary power of modern profiling techniques and emerging bioinformatics tools are being utilized for the discovery of new biomarkers.

Modern metabolite profiling to analyze low-molecular weight analytes such as nucleotides, amino acids, organic acids, sugars or peptides is typically performed by nuclear magnetic resonance (NMR) spectroscopy or tandem mass spectrometry technologies (Jemal et al., 2006, Dettmer et al., 2007, Baumgartner et al., 2007). In particular, quantitative targeted MS-based platforms, using tandem mass spectrometry (MS/MS) coupled with liquid chromatography (LC), allow analysis of metabolites with high sensitivity and structural specificity, and thus minimize potentially confounding clinical variables. However, such platforms still preclude the analysis of large numbers of samples. From the clinical perspective, comparisons of metabolite profiles from quantitative targeted assays in disease versus non-disease states may bring forth novel biomarkers that have the potential to substantially improve cardiovascular diagnostics and support risk prediction of future life-threatening events (Sabatine et al., 2005, Lewis et al., 2008a, 2008b).

In general, the process of searching for biomarkers in biological samples is highly data-driven and dependent on the application of powerful multivariate data mining and statistical bioinformatic methods (Larrañaga et al., 2006, Shulaev, 2006, Osl et al., 2008). Feature selection is a common method for identifying significant variables in multidimensional biomedical data, typically applied prior to classification and biological interpretation. In addition to traditional null-hypothesis significance testing widely-used data mining methods such as filters, wrappers or more powerful meta-learning approaches are used to substantially reduce the dimensionality of data, and allow for the search for those variables that exhibit the best discriminatory ability and prediction. Filters rank variables based on their ability to discriminate predefined cohorts. Here, different entropy-, correlation-, or rule-based evaluation measures such as the information gain, reliefF or associative voting are available to be applied for feature ranking (Hall, 2003, Saeys et al., 2007, Osl et al., 2008). In contrast wrappers use classifiers to evaluate the features' discriminatory ability, exploiting various heuristic paradigms for the search through the feature space to identify reliable predictor subsets, but having the drawback of extensive computational costs. Further improvement in feature selection is achieved by introducing meta-learning models such as embedded or ensemble-based methods (Saeys et al., 2008, Netter et al., 2009).

In biomarker cohort studies a variety of experimental designs are used, from case-control studies to more complex cross-over or serial sampling designs. In particular serial sampling studies allow patients to serve as their own biological controls and permit investigation of kinetic characteristics of circulating analytes by tracking alterations in levels over time. Repeated measure analysis or standard null-hypothesis significance testing are the common methods of making statistical decisions from dependent samples (the null hypothesis H₀ which usually states that two groups do not differ is rejected in favor of an alternative hypothesis H₀ which typically states that the groups differ), using the P-value as an evaluation criterion for the discriminatory ability of variables. Statistical tests calculate whether confidence in a hypothesis based solely on a sample-based estimate exceeds a significance level, but they do not allow for a general valid, clinically relevant categorization of selected metabolites because the P-value is a random variable defined over the sample space (size) of the experiment.

All of the methods described above are not readily applicable to dependent, longitudinal data. Further, biomarkers of early clinical indications, like myocardial injury, are lacking down to the present day.

There is therefore an unmet need for an improved feature selection method, in particular for a method which allows the identification of sensitive, highly predictive discriminatory biomarker candidates participating in metabolic pathways, particularly at early onset of disease.

The present invention addresses this need by providing a method enabling the identification of candidate biomarkers based on their predictive value in paired testing.

According to the invention this need is settled by a method for identification of biomarkers in a subject as it is defined by the features of independent claim 1, by a use of this method as it is defined by the features of independent claim 10 and by a method as it is defined by the features of independent claim 12. Preferred embodiments are subject of the dependent claims.

The method according to the present invention comprises the steps of obtaining a sample from the subject, measuring a plurality of biomolecules in the sample, identifying the measured biomolecules in the sample. The method particularly further comprises the steps of estimating a discriminatory ability of each identified biomolecule by using a paired test hypothesis, and integrating the estimated discriminatory abilities of the biomolecules into a kinetic analysis. Preferably, the subject according to the method of the present invention is animal or human. With such a method, an objective measure for expressing the discriminatory ability (DA) in dependent samples is provided and by using said measure in the kinetic analysis the identification of highly predictive discriminatory biomarker candidates is possible. These identified biomarkers may participate in pathways, which can be associated with diseases. These biomarkers can be used in diagnosis and prognosis of diseases.

Preferably, the sample according to the method of the present invention comprises a body fluid or tissue. Particularly, the body fluid can be blood and urine, blood or urine. Like this, the method of the present invention can be used in clinical testing.

Preferably, the measurement of a plurality of biomolecules is carried out in the sample, preferably by using a profiling platform, which enables a reliable and efficient measuring of said biomolecules.

In one embodiment, the identification of the measured biomolecules is carried out in the sample, wherein the identification is based on the output or results of the previous measurement step.

In one embodiment, the method according to the invention further comprises the step of searching significant correlations and kinetic relations between early- and later-appearing biomolecules in serial sampling studies based on the kinetic analysis.

In one embodiment, the method according to the invention comprises identification and categorization of biomarkers candidates according to their predictive value within the step of estimating the discriminatory ability of each identified biomolecule.

In one embodiment, the method according to the invention comprises within the step of estimating of the discriminatory ability of each identified biomolecule the evaluation of the paired test hypothesis according to the formula:

$$pBI = λ \cdot {DA}^{*} \cdot \sqrt{\frac{\vert {Δ}_{change} \vert}{\vert CV \vert}} \cdot sign ( {Δ}_{change} )$$

$$with$$

$${Δ}_{change} = \{ \begin{matrix}Δ & if Δ \geq 1 \\ - \frac{1}{Δ} & else\end{matrix}$$

wherein pBI is an paired Biomarker Identifier, λ is a scaling factor (e.g. λ=100 by default), DA* is the initial measure DA (e.g in a range of 0.5 to 1) resealed between 0 and 1 weighted by the effect term

$$\sqrt{\frac{\vert {Δ}_{change} \vert}{\vert CV \vert}} ,$$

CV is a coefficient of variation, sign( ) determines the direction of change, Δ_{change} indicates changes in a biomolecule calculated as a relative increase or decrease from the levels of a reference cohort.

Particularly, DA is calculated with a biological effect term as the median percent change in metabolite levels at time point tₓ versus baseline, Δ_{change}, divided by the coefficient of variation (CV) in the normalized data.

In one embodiment, the method according to the invention further comprises that the discriminatory ability of each of the identified biomolecules is estimated by additionally using an unpaired test hypothesis. Like this, the method can be applied to both dependent and independent samples to overcome the problem of interpreting ‘relative’ P-values that exceed or not exceed significance levels when applying statistical null-hypothesis testing, to reveale higher performance than statistical testing and other feature selection methods for ranking variables according to their predictive value, and to further provide a reliable biomarker categorization scheme of high diagnostic and prognostic relevance.

Particularly, the unpaired test hypothesis can be evaluated within the method of the invention according to the formula:

$$uBI = λ \cdot {TP}^{{2}^{*}} \cdot \sqrt{\vert {Δ}_{change} \vert \frac{{CV}_{ref}}{CV}} \cdot sign ( {Δ}_{change} )$$

$${Δ}_{change} = \{ \begin{matrix}Δ & if Δ \geq 1 \\ - \frac{1}{Δ} & else\end{matrix} \\ with \\ {Δ}_{ref} = \frac{\overline{x}}{\overline{x}}$$

wherein uBI is an unpaired Biomarker Identifier, λ is a scaling factor (e.g. λ=100 by default), TP²* is an initial measure defined as TP² (e.g. with a range between 0.25 to 1) resealed between 0 and 1, Δ_{change} indicates changes in biomolecules calculated as a relative increase or decrease from the levels of a reference group, CV is a coefficient of variation, CV_{ref} is a reference coefficient of variation, sign( ) determines the direction of change, x̅ is the mean value from the levels of a biomolecule in a cohort, x̅_{ref} is the mean value from the levels of a biomolecule in a reference cohort. Particularly, TP²* denotes the discriminatory ability of a metabolite determined from logistic regression analysis.

Analogous to DA, the discriminance measure TP²* is weighted by a biological effect term

$$\sqrt{\vert {Δ}_{change} \vert \frac{{CV}_{ref}}{CV}} . ,$$

comprising the parameters Δ_{change} and CV/CV_{ref}. Δ_{change} is divided by the quotient CV/CV_{ref}.

All of these embodiments according to the method of the invention contribute to provide an efficient bioinformatic tool for biomarker identification.

Preferably, the biomolecules according to the method of the invention are selected from a group consisting of nucleotides, amino acids, organic acids, sugars, lipids, acyl-camitines, peptides or proteins.

Another aspect of the invention relates to the use of the method described above for risk prediction of future life threatening events in disease, prognosis or diagnosis.

Still another aspect of the invention relates to a method for monitoring progress or treatment of a disease, comprising the steps of (a) providing numerical scores for biomarkers based on the discriminatory ability by using the method for identification of biomarkers according to the invention, wherein the scores are predetermined to be relevant to the disease, (b) repeating step (a) after a period of time during which subjects receive treatment for the disease, to obtain post-treatment scores; (c) comparing the post-treatment scores from step (b) with the scores in step (a) versus scores for subjects not suffering under the disease, and (d) classifying said treatment as being effective if scores from step (b) are closer to the scores from step (a) than to the scores for normal subjects.

Advantageously, the method for monitoring progress or treatment of a disease can be used as an efficient bioinformatic tool for biomarker discovery, for example, in clinical metabolomics in order to aid in diagnostics, prognosis and risk prediction of metabolic diseases, such as cardiovascular diseases. By applying the method of the present invention in a clinical field, it is possible to identify a series of metabolites participating in pathways tightly associated, for example, with diseases, particularly myocardial infarction or human cardiovascular disease in general, some of which changed as early as 10 minutes after injury, a time frame in which no currently available clinical biomarkers are present.

In a particular embodiment, the method for monitoring progress or treatment of a disease is further characterized in that the numerical scores for biomarkers is carried out in a diseased cohort (cases) versus a healthy cohort (controls) and/or in longitudinal biomarker cohort studies (e.g. times series).

In one embodiment, the period of time according to the method for monitoring progress or treatment of a disease is within a range of minutes, preferably 1, 2, 3, 4, 5, 6, 7, 8, 9, 10, 20, 30, 40, 50, 60, 120 or 240 minute(s), hours, preferably 1, 2, 3 or 4 hour(s), days, preferably 1, 2, 3, 4, 5, 6, 7, 8, 9, 10, 10 to 20, 20 to 28, 20 to 29, 20 to 30, 20 to 31, 20, 28, 29, 30, 31 day(s) or months, preferably 1, 2, 3, 4, 5, 6, 7, 8, 9, 10, 11 or 12 month(s) after the onset of said disease or treatment. The disease, disorder or defect according to the methods of the invention and their uses can be selected from the group consisting of metabolic diseases or cancer. Particularly, the defect, disorder or disease is correlated or indicated with amino acid metabolism (e.g. phenylketonuria, urea cycle), mitochondrial fatty acid oxidation (e.g. medium chain acyl dehydrogenase deficiency (MCADD), long chain acyl CoA dehydrogenase deficiency (LCAD), very long chain acyl CoA dehydrogenase deficiency (VLCAD), carnitine cycle defects, lipid storage disease), metabolism of carbohydrate (e.g. defects in intermediary carbohydrate metabolism, defects in galactose metabolism, defects in fructose metabolism, defects in intermediary carbohydrate metabolism associated with lactic acidosis such as hypoxic organ damage (e.g. stroke, myocardial infarction), glycogene storage diseases), mucopolysaccharide metabolism, metabolism of purines and pyrimedines, porphyrias consisting of acute (hepatic) porphyrias and cutaneous (erythropoietic) porphyrias, hypoglycemia (e.g. diabetes), cancer such as prostate cancer, ovarian cancer, metabolic changes in neoplastic disorders such as albinism, alkaptonuria, amyloidosis, chediak-higashi syndrome, cystinosis, fabry's disease, galactosemia, gaucher's disease, gout, hernochromatosis, histiocytosis, homocystinuria, lipidoses, marfan's syndrome, marchesani's syndrome, mucopolysaccharidosis, niemann-pick disease, osteogenesis imperfecta, wilson's disease or metabolic diseases including cardiovascular disease such as western life style disorders like metabolic syndrome.

Still another aspect of the invention relates to a computing system such as, e.g., a processor-based desktop computer or a processor based computing apparatus integrated into another device (embedded system). The system comprises computing means (processor) and memory means which are arranged to perform at least part of the steps of the methods according to the invention. In particular, the computing system comprises means for automatically estimating the discriminatory ability of each identified biomolecule by using paired test hypothesis and means for integrating the estimated discriminatory abilities of the biomolecules into a kinetic analysis. With such a computing system, the provision of an objective measure for expressing the discriminatory ability (DA) in dependent samples can be efficiently performed.

Preferably, the computing means and the memory means of the computing system are arranged to perform further steps of the methods according to the invention as described above. Also, the computing system can comprise interface means for interacting with other devices, such as for example a device for measuring the plurality of biomolecules in the sample, a device for identifying the measured biomolecules in the sample or input/output devices.

Still another aspect of the invention relates to a computer program which is arranged to perform at least part of the steps of the methods according to the invention when it is run on a computing system as mentioned above.

### DEFINITIONS

The technical terms and expressions used within the scope of this application are generally to be given the meaning commonly applied to them in the pertinent art of bioinformatic.

The term “subject” as used within the present invention relates to an animal, human, eukaryotic or prokaryotic cells or complex biological mixtures. Further, the term “subject” refers to any warm-blooded animal, particularly including a member of the class mammalia such as, without limitation, humans and non-human primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex and, thus, includes adult and newborn subjects, whether male or female.

As used herein, “treatment” refers to ameliorating an adverse heart condition such as myocardial ischemia or myocardial infarction. As used herein, “detecting” refers to methods which include identifying the presence or absence of substance(s) in the sample, quantifying the amount of substance(s) in the sample, and/or qualifying the type of substance. “Detecting” likewise refers to methods which include identifying the presence or absence of myocardial ischemia or early myocardial infraction in a subject. “Mass spectrometer” refers to a gas phase ion spectrometer that measures a parameter that can be translated into mass-to-charge ratios of gas phase ions. Mass spectrometers generally include an ion source and a mass analyzer. Examples of mass spectrometers are time-of-flight, magnetic sector, quadrupole filter, ion trap, ion cyclotron resonance, electrostatic sector analyzer and hybrids of these. “Mass spectrometry” refers to the use of a mass spectrometer to detect gas phase ions.

The term “normal subjects” relates to a subject as defined herein above not suffering under a disease.

The term “sample” as used herein refers to a “biological sample”, meaning a sample obtained from a subject. The biological sample can be selected, without limitation, from the group consisting of blood, plasma, serum, sweat, saliva, including sputum, urine, and the like. As used herein, “serum” refers to the fluid portion of the blood obtained after removal of the fibrin clot and blood cells, distinguished from the plasma in circulating blood. As used herein, “plasma” refers to the fluid, noncellular portion of the blood, distinguished from the serum obtained after coagulation.

The term “discriminatory ability”, as used herein, refers to the assessment of the discriminatory ability of variables to distinguish between two independent samples (e.g. cases versus controls), an established measure of a diagnostic test is the area under the receiver operating curve (AUC). It incorporates sensitivity and specificity as the two main features of the test. Alternatively, the product of sensitivity and specificity can be used as a further accepted feature of a diagnostic test. The discriminatory ability is defined as the percent change of biomolecule concentration levels in a cohort in one direction versus baseline (dependent samples).

The term “paired biomarker identifier” (pBI), as used within the present invention, relates to univariate feature selection variant for the search and prioritization of metabolic signatures based on a paired test hypothesis.

The term “unpaired biomarker identifier” (uBI) defines an univariate feature selection variant for the search and prioritization of metabolic signatures based on an unpaired test hypothesis. The uBI can be used in an independent test setting, like case-control studies.

As used herein, the term “kinetic analysis” means the investigation of the dynamic characteristic of circulating biomolecules in the body over time.

The term “weak, moderate strong predictor” refers to biomarker candidate classified according to its discriminatory ability into weak, moderate strong predictors.

The term “numerical scores for selected biomarker (candidates)”, as used herein, relates to absolutes score values delivered from the method variants “paired” and “unpaired biomarker identifier” on numerical data sets (e.g. metabolite concentrations).

As used herein, the term “profiling platform” relates to analytical devices like nuclear magnetic resonance (NMR) spectroscopy or tandem mass spectrometry, targeted mass spectrometry (MS) or quantitative mass spectrometry (MS), which is enabled by coupling tandem mass spectrometry (MS/MS) with liquid chromatography (LC).

The term “complex biological mixtures” can be, as used herein, for instance blood, urine or tissue.

The term “prognosis”, as used herein, has a medical meaning and describes the likely outcome of a defect, disease or disorder, which are particularly defined within the embodiments of the present invention.

The term “diagnosis”, as used herein, is a label given for a medical condition, disease, disorder or defect identified by its signs, symptoms, and from the results of various diagnostic procedures. The term “diagnosis” includes the recognition of a disease, disorder, defect or condition by its outward signs and symptoms as well as the analysis of the underlying physiological/biochemical cause(s) of a disease, disorder, defect or condition. The term “diagnostic criteria” designates the combination of signs, symptoms, and test results that allows the doctor to ascertain the diagnosis of the respective disease.

The term “risk prediction of future life threatening events” defines a particular event like a disease, disorder or defect that will occur in the future in more certain terms than a forecast.

As used herein, the term “biomarker” or “biomarker candidate” defines a substance used as an indicator of a biological state. It is a characteristic that is objectively measured and evaluated as an indicator of normal biological processes, pathogenic processes, or pharmacologic responses to a therapeutic intervention. Biomarkers are characteristic biological properties that can be detected and measured in parts of the body like the blood or tissue. They may indicate either normal or diseased processes in the body. Biomarkers can be specific cells, molecules, or genes, gene products, enzymes, or hormones. Complex organ functions or general characteristic changes in biological structures can also serve as biomarkers.

The term “disease cohort” is defined as a group of subjects who have shared a particular abnormal genetic profile or clinical picture during a particular time span.

The term “healthy cohort” is defined as a group of subjects who have shared physical health or fitness during a particular time span.

The term “longitudinal biomarker cohort studies is a correlational research study of the same subjects (cohort) that involves repeated observations of the same biomolecules (biomarkers) over long periods of time.

The term paired test hypothesis relates to the paired or “dependent samples” test hypothesis and is used when dependent samples typically consist of a sample of matched pairs of similar units, or one group of units that has been tested twice (“repeated measures”).

The term unpaired test hypothesis relates to the unpaired or “independent samples” test hypothesis and is used when two separate independent and identically distributed samples are obtained, one from each of the two populations being compared.

The term “level” stands e.g. for biomolecule (metabolite) concentration.

The present invention is further described by reference to the following non-limiting figures, tables and examples.

The figures show:

FIG. 1 illustrates ROC curves and AUCs estimated for pBI versus paired statistical hypothesis testing. The cut-offs DA*=0.2 (weak predictors, see figure), DA*=0.4 and 0.6 (moderate and strong predictors, ROC curves not shown) has been used in order to define the dependent variable for ROC analysis. The inverse P-values and absolute uBI scores were used in this analysis.

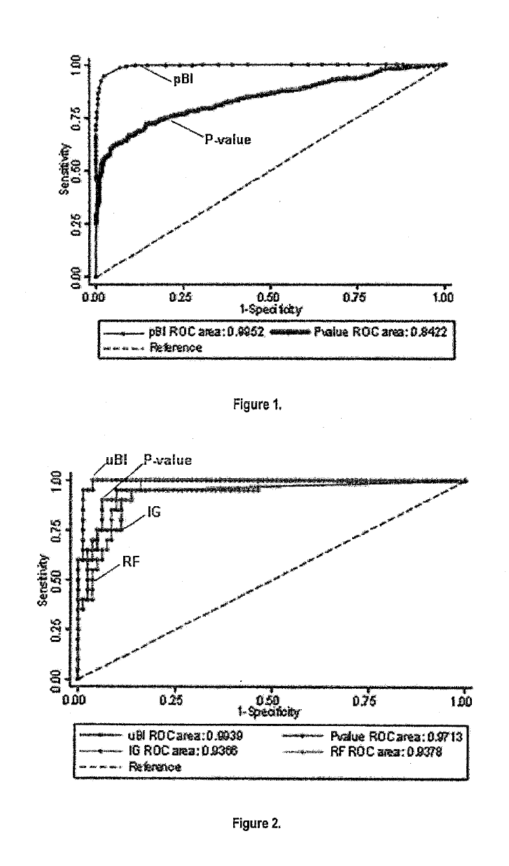

FIG. 2 shows ROC curves and AUCs estimated for uBI versus unpaired statistical testing (P-value), IG and RF are depicted. TP² (TP²*) is set to 0.4 (0.2) for defining the weak predictor class. TP² is the product of sensitivity and specificity. The inverse P-values and absolute uBI scores were used in this analysis.

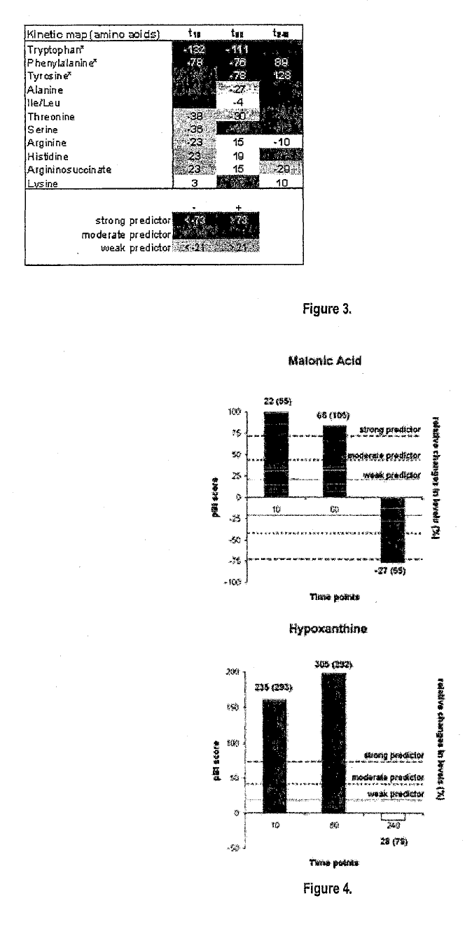

FIG. 3 shows the kinetic map of amino acids on PMI data at 10, 60, and 240 minutes after myocardial injury using the pBI scores. In general, red color increments indicate decreasing levels, blue color increments indicate increasing levels. The colors in FIG. 3 correspond to the FIG. 3 color legend as follows:

| | | | |
|---|---|---|---|
| dark red: | <−73 | dark blue: | >73 |
| medium red: | <−44 | medium blue: | >44 |
| light red: | <−21 | light blue: | >21 |

The metabolites tryptophan*, phenylalanine* and tyrosine* are associated with cardiac catheterization alone.

FIG. 4 illustrates the kinetic characteristic of malonic acid and hypoxanthine: Categorization of pBI scores across the time points at 10, 60, and 240 minutes after PMI are exemplarily shown (height of bars). Values above bars denote median (IQR) of relative changes in levels versus baseline in percent.

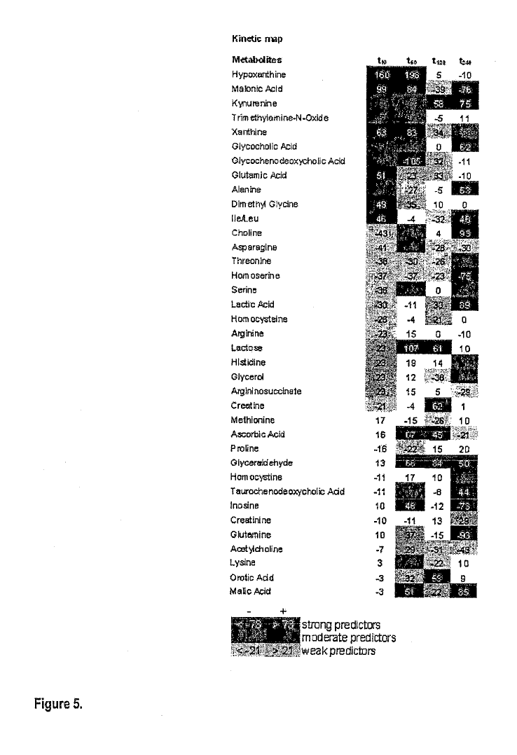

FIG. 5 shows the kinetic map of selected metabolites in PMI after data preprocessing using the pBI model. Scores are sorted by column t₁₀ (10 minutes) in descending order, indicating early markers in PMI.

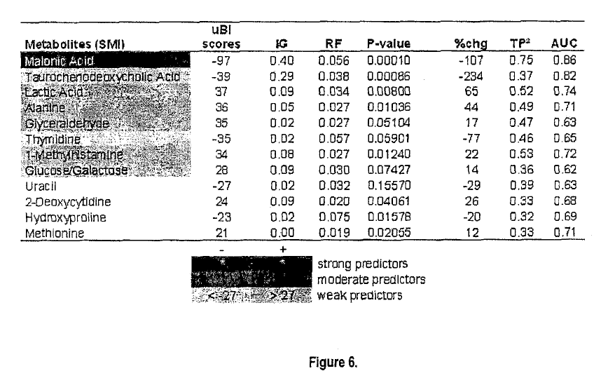

FIG. 6 shows the prioritization of metabolic signatures in SMI using the methods uBI, IG, RF and statistical significance testing (P-value). The symbol % chg denotes the median percent change in metabolite levels in patients with SMI versus patients undergoing diagnostic cardiac catheterization with nonacute cardiovascular disease (controls). The last two columns denote the discriminatory ability of metabolites expressed by the product of sensitivity and specificity (TP²) and the AUC. The table includes all metabolites with absolute % chg ≧10 (calculated on the initial data set). Results for uBI, IG, RF, P-values, TP² and AUC were 10-fold cross validated.

The foregoing description will be more fully understood with reference to the following Examples. Such Examples, are, however, exemplary of methods of practicing the present invention and are not intended to limit the scope of the invention

The following Examples illustrate the invention:

### EXAMPLE 1

### Material and Methods

### Targeted LC-MS/MS Metabolite Profiling

The metabolites have been incorporated in the platform that follow criteria of biological diversity and relevance, covering as many of the known metabolic pathways in cardiovascular disease and in human metabolism in general. Metabolites were excluded if they were not readily detectable by LC-MS/MS analysis. The detailed protocol of MS analysis was recently published in www.jci.org (Lewis at al., 2008). Briefly, blood samples were drawn from peripheral femoral venous catheters during the procedure and collected in K2EDTA-treated tubes. Samples were immediately centrifuged and the supernatant plasma was aliquoted for separation using high performance liquid chromatography (HPLC). Three HPLC columns for separating sugars and ribonucleotides, organic acids, and amino acids were aligned in sequence with a triple quadrupole mass spectrometer (AB4000Q, Applied Biosystems, MA) using a turbo ion spray LC/MS interface. Targeted MS/MS analysis using selective reaction monitoring (SMR) conditions was performed allowing monitoring a total of 210 metabolites for each sample. Targeted MS methods using MS/MS coupled with LC permit highly specific identification of analytes. Addition of isotope labeled internal standards for selected metabolites enabled absolute measurements of analyte concentrations by integrating peak areas for parent-daughter ion pairs in MS/MS spectra.

A Clinical Model of Planned Myocardial Infarction

A clinical model of planned myocardial infarction (PMI) was chosen that faithfully represents spontaneous myocardial infarction to study kinetics of small molecules in human plasma over time. The targeted metabolite profiling to serial blood samples obtained from patients undergoing alcohol septal ablation for hypertrophic obstructive cardiomyopathy (HOCM) have been applied. Serial sampling before (baseline) and, according to the protocol, at 10 minutes, 1 hour, 2 hours, and 4 hours after injury allowed patients to serve as their own biological controls and permitted kinetic analyses of circulating metabolites, particularly at early stage after injury. A total of 31 patients who underwent alcohol septal ablation for the treatment of symptomatic HOCM were enrolled within the present invention. The number of samples, however, partly differed between time points at 10 minutes (N=31), 1 hour (N=28), 2 hours (N=25), and 4 hours (N=11). Inclusion criteria as well as detailed patient characteristics can be found in Lewis et al., 2008. The protocol was approved by the Massachusetts General Hospital Institutional Review Board, Boston, Mass. and all individuals gave written informed consent.

Patients with Spontaneous Myocardial Infarction

A cohort of 12 patients undergoing emergent cardiac catheterization for acute spontaneous ST-segment elevation myocardial infarction (SMI) was enrolled within 8 hours of symptom onset. Blood samples were drawn during emergent catheterization. A second cohort of 9 patients undergoing elective, diagnostic cardiac catheterization for cardiovascular disease without acute symptoms of myocardial ischemia was included within the present invention as a control cohort. In both groups blood was drawn prior to the onset of catheterization and at 10 minutes and 60 minutes after the procedure was completed. A total of 26 samples from multiple time points in SMI and 18 samples in controls were included. Note that samples from all time points were not available for MS analysis.

Computational Approach for Biomarker Search, Prioritization and Dynamic Analysis

Data Preprocessing

An extensive review of generated LC-MS data to specified criteria and laboratory action items was performed to ensure a high level of reliability, completeness, reproducibility and consistency in the data. Raw data have been carefully examined, determining if all requested metabolites were accounted for, if targeted measurement levels were met and then correlated data of historical experiments to ensure that analyte levels were stable over time. A three step preprocessing procedure on technically validated data has been applied in order to provide quality assured data sets for the purpose of biomarker search; 1) Of the 210 measured metabolites with more than 20% missing values in the data set (for each cohort separately) have been excluded to ensure relatively uniform conditions for statistical analysis. 2) To avoid distortion of statistical results, outlier detection was performed. A common statistical model has been applied using the interquartile ranges (IQRs), defining an outlier as observation outside the range [Q₁−k·IQR; Q₃+k·IQR], with Q₁, Q₃=first and third quartiles, IQR=Q₃−Q₁. k=3 was set to only remove ‘extreme’ outliers in the data. In the present invention, 4.4% of MS values in PMI and 1.2% in SMI were detected as strong outliers and removed from the data sets using this approach. 3) For kinetic analysis, serial PMI data (5 time points) have been normalized to the metabolites' baseline levels, denoted in percent changes.

Prioritization Model for Paired and Unpaired Dichotomous Samples

A new univariate feature selection model for the search and prioritization of metabolic signatures using serial PMI and SMI data was developed.

1. The Model's Variant Addressing the Paired Test Problem:

Because main features of a diagnostic test (sensitivity, specificity or the area under the receiver operating curve (AUC)) are not defined for paired testing, a new, objective measure for expressing the discriminatory ability (DA) in dependent samples have been introduced. DA is defined as the percent change of metabolite levels in a cohort in one direction versus baseline. As an example, if 50% of cohort subjects yield increasing or respectively decreasing metabolite levels, the discriminatory ability DA is calculated to be 0.5 (this value corresponds exactly to AUC=0.5 in unpaired testing), denoting no discrimination. If 75% or 100% of subjects show increasing or respectively decreasing levels, DA=0.75 or 1.0, indicating good or perfect discrimination.

Based on this definition the model's variant pBI, termed paired Biomarker Identifier have been developed that combines the introduced discriminance measure DA with a biological effect term calculated as the median percent change in metabolite levels at time point tₓ versus baseline, Δ_{change}, divided by the coefficient of variation (CV) in the normalized data:

$$pBI = λ \cdot {DA}^{*} \cdot \sqrt{\frac{\vert {Δ}_{change} \vert}{\vert CV \vert}} \cdot sign ( {Δ}_{change} ) , \\ {Δ}_{change} = \{ \begin{matrix}Δ & if Δ \geq 1 \\ - \frac{1}{Δ} & else\end{matrix} \qquad ( 1 )$$

λ is a scaling factor (λ=100 by default), DA* is the initial measure DA (range: 0.5-1) rescaled between 0 and 1, weighted by the effect term

$$\sqrt{\frac{\vert {Δ}_{change} \vert}{\vert CV \vert}} .$$

Note that a CV greater than 1 is set to 1 by default to consider solely data distributions with smaller variance in normalized data to be interpreted as a positive biological effect. The function sign( ) determines the direction of change. In summary, pBI is built on four statistical determinants that are the discriminatory ability DA, magnitude, variance and direction of changes in metabolite levels, permitting a biologically feasible prioritization of metabolic signatures, as it is proposed, into weak, moderate and strong predictors.

To evaluate the power of feature selection, pBI is benchmarked with a paired, one sample, two tailed significance hypothesis test (Student t-test or Wilcoxon signed-rank-test, the latter if the population is not normally distributed). There are no adequate feature selection methods to be compared with, operating on a paired test hypothesis.

2. The Model's Variant Addressing the Unpaired Test Problem:

To distinguish between two independent populations the product of the true positive rates of both classes (TP²) have been determined as an objective measure for discrimination. For calculating TP² classifiers like support vector machines or logistic regression are used, the latter especially in a biomedical setting (Cristianini et al., 2000, Hosmer et al., 2000). In a diagnostic test, TP² is defined as the product of sensitivity and specificity which is an accepted diagnostic parameter tightly associated with the AUC, denoting the probability of correctly classifying a randomly selected true positive and true negative subject. The product is now to be interpreted as follows: If sensitivity and specificity is 0.5, TP²=0.25, indicating no valuable discrimination (cf. DA=0.5, paired testing). To guarantee true positive and true negative values ≧0.5, TP²=0 was set if either sensitivity or specificity is <0.5, thus indicating no discriminatory value, while TP²=1.0 depicts perfect discrimination. Analogous to pBI, the variant uBI, termed unpaired Biomarker Identifier was developed which is defined as:

$$uBI = λ \cdot {TP}^{{2}^{*}} \cdot \sqrt{\vert {Δ}_{change} \vert \frac{{CV}_{ref}}{CV}} \cdot sign ( {Δ}_{change} ) , \\ {Δ}_{change} = \{ \begin{matrix}Δ & if Δ \geq 1 \\ - \frac{1}{Δ} & else\end{matrix} \\ with \\ Δ = \frac{\overline{x}}{{\overline{x}}_{ref}} \qquad ( 2 )$$

λ is a scaling factor (λ=100 by default). TP²* is the initial measure TP² (range: 0.25-1) rescaled between 0 and 1, and denotes the discriminatory ability of a metabolite determined from logistic regression analysis in this study (Homser et al., 2000). Analogous to DA, the discriminance measure TP²* is weighted by a biological effect term

$$\sqrt{\vert {Δ}_{change} \vert \frac{{CV}_{ref}}{CV}} ,$$

comprising the parameters Δ_{change} and CV/CV_{ref}. The symbol Δ_{change} indicates changes in metabolites calculated as a relative increase or decrease from the levels of a reference group (controls) and is divided by the quotient CV/CV_{ref}, denoting changes in the variance of data across the two cohorts where CV<CV_{ref} is interpreted as a positive biological effect. CV/CV_{ref} values greater than 1 are set to 1 (see also pBI). The function sign( ) determines the direction of change. It should be noted that through rescaling the initial parameters DA and TP² the absolute pBI and uBI scores permit a high degree of comparability and generalizability between both feature selection modalities.

uBI was benchmarked with two widely used feature selection methods that are the information gain IG (the IG of a feature aᵢ reflects how much information the feature provides on the class attribute cⱼ, and is calculated by IG(aᵢ)=E(cⱼ)−E(cⱼ|aᵢ), where E(cⱼ) is the entropy of the class cⱼ and E(cⱼ|aᵢ) is the conditional entropy of cⱼ given aᵢ) and reliefF (RF), an instance based, multivariate feature selection method built on the assumption that useful features have significantly different values for instances of different classes and similar values for instances of the same class as well as standard statistical hypothesis testing using an unpaired, two sample, two tailed null-hypothesis significance test (Student t-test or Mann-Whitney-U test if the population is normally versus not normally distributed) (Hall, 2003, Osl et al., 2008).

Kinetic Mapping

For visualizing dynamic changes in metabolite levels a 2D pseudo color representation was employed on serial pBI scores calculated from given metabolites according to the proposed scheme of weak, moderate and strong predictors.

ROC Analysis to Estimate the Discriminatory Ability of Selected Metabolites

To assess the predictive ability of variables to distinguish between two independent samples (e.g. cases versus controls), an established measure of a diagnostic test is the area under the receiver operating curve (AUC) (Fawcett, 2006). It incorporates sensitivity and specificity as the two main features of the test. In the ROC curve, specificity is usually denoted as 1—specificity. An AUC of 1 depicts a test with perfect discrimination, whilst an AUC of 0.5 denotes an uninformative test (45° diagonal line in the graph). Alternatively, the product of sensitivity and specificity can be used as a further accepted feature of a diagnostic test. Within the present invention ROC analysis was also used to compare different feature selection methods and to estimate their power to rank variables according to their discriminatory ability. A χ² statistics was applied for testing differences between ROC curves being statistically significant.

Cross-Validation to Generalize Findings

Because PMI and SMI cohorts are of small size, a cross validation strategy was applied to estimate the degree of reliability of findings on a single derivation cohort. In classification stratified 10-fold cross validation is an accepted statistical practice of partitioning a sample of data into ten subsets where each subset is used for testing and the remainder for training, yielding an averaged overall error estimate. For feature selection the data set is also subdivided into ten partitions. The process is repeated 10 times, using 9 partitions for generating the feature ranking where rankings of each partitioning are finally aggregated and expressed by a mean±SD rank (Witten et al., 2005).

### EXAMPLE 2

### Discriminatory Ability (DA) and Categorization of Metabolites

Discriminatory Ability (DA) and Categorization of Metabolites Using pBI Scores

A new discriminatory measurement (DA) was applied to paired testing in order to define three classes of biomarkers of planned myocardial infarction: Weak predictors, defined by a DA cut-off of 0.6 (selected metabolites with DA<0.6 have less or no predictive value), moderate predictors defined by the cut-offs of 0.7 and 0.8 (0.7≦DA<0.8), and strong predictors equal to or above the threshold DA=0.8. Using the transformed measure DA* as implemented in eq. 1, rescaled DA cut-offs (i.e. DA*=0.2, 0.4 and 0.6) thus allow direct comparison with the corresponding measure TP²* defined for independent samples.

The strength of the methods pBI versus paired Student t-test/Wilcoxon signed-rank-test was estimated to select predictors using ROC analysis. DA cut-offs as described above were used to define the dependent variable (dichotomous), and uBI scores and P-values calculated for analytes at all 5 time points served as independent variables. A total of 173 metabolites, comprising sugars, ribonucleotides, organic and amino acids, were included in this analysis after data preprocessing as described in Methods. Subsequently standard logistic regression analysis was applied to calculate the pBI score thresholds for the proposed categorization scheme. Table 1 and FIG. 1 show the detailed results. It can be seen that AUC values ≦0.994 for DA*=0.2, 0.4 and 0.6 underscore the expected high performance of the method pBI. It was the objective within the present invention to design a new scoring model that is tightly associated with the discriminance measure DA (DA*), integrating additional metabolic information expressed by the magnitude, variance and direction of metabolite changes, and thus permitting feasible prioritization of candidate biomarkers identified in longitudinal studies. Using paired significance testing, AUC values (0.94, 0.91, 0.84) were statistically significantly lower than pBI values (χ² statistics, P<0.001, Table 1), confirming that pBI better ranks variables with regard to their predictive value compared to P-value ratings.

As shown in Table 1, the pBI score thresholds were estimated as follows: 21+ for classifying weak, 44+ for moderate, and 73+ for strong predictors. It should be noted that pBI may also allow a further prioritization of metabolites beyond the cut-off for ‘strong’ predictors, however relying on the general discriminatory ability of analytes determined by the complexity of the underlying metabolic pathways. For example in less intricate (monogenic) diseases such as inborn errors of metabolism scores beyond 100 or 500 in small sets of metabolites are more likely, and are therefore termed as ‘key’ or ‘primary’ markers, showing extremely elevated levels (up to 10-100 fold higher levels versus reference), and DA or TP² values close to 1.0 (Baumgartner et al., 2006).

Discriminatory Ability and Categorization of Metabolites Using uBI Scores

Analogously to pBI, uBI's power was compared to select variables with an unpaired Student t-test/Mann Whitney-U-test, and two popular entropy- and correlation-based feature selection methods, that are the IG and RF using the SMI data (Table 2). Estimating the model's performance for weak predictors (TP²*=0.2), uBI revealed the largest AUC, achieving statistical significance (P=0.0269) compared with the other methods. For this predictor class an uBI score threshold of 27 was estimated that is only 6 score points higher than for pBI. Using TP²*=0.4, uBI also revealed the largest AUC for the moderate predictor class, however without achieving statistical significance. The score cut-off for this class was calculated to be 50 (cf. 44 in pBI).

TABLE 1
Power of pBI and score cut-offs for
analyte categorization on PMI data

| | | Strong | Moderate | Week |
|---|---|---|---|---|
| | | predictors | predictors | predictors |
| DA (DA*) | cut-off | 0.8 (0.6) | 0.7 (0.4) | 0.6 (0.2) |
| \|pBI\| score | cut-off | 73 | 44 | 21 |
| ]pBI\| score | AUC | 0.994 | 0.996 | 0.995 |
| P-value | AUC | 0.943 | 0.912 | 0.842 |
| H₀: area(pBI) = | χ² | 27.5 | 63.1 | 145.5 |
| area(P-value) | Prob > χ² | 0.0000 | 0.0000 | 0.0000 |

Table 1 illustrates the ROC analysis for estimating the power of the method pBI versus paired statistical hypothesis testing (P-value) to select variables according to their predictive value. A χ² test was applied to determine statistical significance between ROC curves. Logistic regression analysis was used to estimate pBI score cut-offs for classifying metabolites as weak, moderate and strong predictors. A total of 878 data points, comprising 173 metabolites×5 time points (excluding 15 NaN values) were used for ROC analysis.

Not enough data was available beyond TP²*=0.6 to carry out this analysis. Assuming a positive intercept of roughly 5 score points between PMI and SMI cut-offs, it seems to be likely that the threshold for strong predictors lies in the range of 75 and 80. However, despite the limited data available for this analysis, uBI yielded the best performance of ranking variables with respect to their discriminatory ability for both the weak and moderate predictor class. The corresponding ROC curves for the weak predictor class are depicted in FIG. 2.

TABLE 2
Power of uBI and score cut-offs for
analyte categorization on SMI data

| | | Strong | Moderate | Week |
|---|---|---|---|---|
| | | predictors | predictors | predictors |
| TP² (TP²*) | cut-off | 0.7 (0.6) | 0.55 (0.4) | 0.4 (0.2) |
| \|uBI\| score | cut-off | — | 50 | 27 |
| \|uBI\| score | AUC | — | 0.990 | 0.994 |
| P-value | AUC | — | 0.980 | 0.971 |
| IG | AUC | — | 0.980 | 0.937 |
| RF | AUC | — | 0.857 | 0.938 |
| H₀: area(uBI) = | χ²(3) | — | 1.06 | 9.19 |
| area(P-value) = | Prob > χ² | — | 0.7856 | 0.0269 |
| area(IG) = area(RF) | | | | |

Table 2 illustrates the ROC analysis for estimating the power of the method uBI versus unpaired statistical testing (P-value), IG and RF to rank variables according to their predictive value. A total of 102 metabolites were considered for analysis to estimate the uBI cut-offs for categorization. Too little data was available to perform a representative ROC analysis for TP²*=0.6.

The proposed data mining method aims at enhancing the predictive power of selected analytes by combining objective measures of discrimination such as DA or TP² with metabolic determinants expressed by changes in magnitude, variance and direction in metabolite levels versus baseline (pBI) or versus an independent reference group (uBI).

### EXAMPLE 3

### Kinetic Analysis of Metabolites in PMI

Peripheral blood samples in a cohort of 31 patients were studied to analyze alterations in analyte levels across five time points. FIG. 3 shows a 2D pseudo color plot of pBI scores for a selected group of metabolites (amino acids) at 10 minutes (t₁₀), 1 hour (t₆₀), 2 hours (t₁₂₀), and 4 hours (t₂₄₀) after myocardial injury. Scores are sorted by column t₁₀ in descending order to focus on the investigation of early-appearing biomarker candidates. A 2D plot of additional interesting metabolites is shown in FIG. 5. Within the class of amino acids e.g. alanine showed decreased concentration levels ≦60 minutes, while other early metabolites like threonine and serine changes persisted between 10 and 240 minutes after injury. Interestingly, isoleucine/leucine (Ile/Leu) yielded increased levels as early as 10 minutes, but decreased levels >60 minutes after injury, and again changes in levels with reverse direction beyond 240 minutes. According to the pBI score scheme all these metabolites revealed moderate predictive value. Tryptophan, phenylanaline and tyrosine classified as strong predictors at the 10-minute and 60-minute time point appeared to be promising biomarkers candidates. However, these metabolites also changed with cardiac catheterization alone, indicating their lack of specificity for myocardial injury, and therefore were excluded from further analysis.

Further candidates of early-appearing metabolites include products of purine and pyrimidine catabolism (ATP, ADP, hypoxanitine, xanitine and malonic acid) being classified as strong predictors, trimethylamine N-oxide (TMNO) which is associated with injury-mediated modulation of dietary compounds, kynurenine, and a spectrum of weak predictors including inosine, which has been shown to reduce cardiomyocyte apoptosis (Goldhaber et al., 1982, Bäckström at al., 2003). FIG. 4 singles out the dynamic characteristic of malonic acid and hypoxanthine in a box plot as well as a bar graph according to the pBI scoring categorization scheme.

A serial sampling design in PMI was chosen, first to search for correlations and kinetic relations between early- and later-appearing metabolites, thus giving a deeper insight into kinetic mechanisms of metabolites involved in pathways associated with myocardial injury. Secondly, due to the profound degree of interindividual variability observed in multiple cohort studies, and limitations of the technology that still suffers from moderate signal-to-noise ratios, serial sampling studies overcome these restrictions because each patient serves as his or her own biological control which significantly reduces the platform-based variability.

Presently used indicators of myocardial injury such as cardiac troponin or the myocardial isoform of creatine kinase are not readily detectable until at least 4 hours after myocardial injury (Zimmerman at al., 1999). Within the present invention, significant metabolite alterations in the PMI cohort were identified that were unambiguously apparent as early as 10 minutes after injury and validated using a stratified cross validation strategy. The scoring model was used to visualize and prioritize dynamic changes across a spectrum of time points using a 2D pseudo color representation which allows for a quick and extensive review for kinetic patterns inherent in the data.

Thus, changes in metabolites that had been previously reported including sequential purine degradation products (ADP, ATP, hypoxanthine, xanthine), pyrimidine metabolites including malonic and aminoisobutyric acid, intermediates of anaerobic glycolysis (lactic acid), and multiple amino acids (Goldhaber at al., 1982, Mei et al., 1996, Zimmerman et al., 1999, Turgan at al., 1999, Bäckström et al., 2003) have been confirmed within the present invention.

### EXAMPLE 4

### Prioritization of Metabolic Markers in SMI

The uBI model was evaluated on data provided from patients with SMI presenting for acute coronary angiography versus patients undergoing elective, diagnostic cardiac catheterization without acute coronary syndromes for myocardial ischemia serving as controls. For this analysis a total of 102 metabolites after data preprocessing were available. FIG. 6 shows the prioritized list of metabolites using the uBI score scheme, the values obtained from the methods IG, RF and unpaired statistical testing, the relative increase or decrease of metabolite levels in SMI from those of the reference cohort expressed as median percent change, as well as the product of sensitivity and specificity (TP²) and AUC for each single analyte. Interestingly, malonic acid alone, which is a product of the pyrimidine catabolism, revealed highest predictive value expressed by an AUC=0.86 and a product of 0.75 (cross-validated), and thus is classified as strong predictor. Lactic acid, a metabolite related to myocardial anaerobic metabolism, showed a high AUC value of 0.74 as well, and was classified as a moderate predictor.

### EXAMPLE 5

### Validation of Metabolic Markers in SMI

The exact time of onset of SMIs relative to sample collection was heterogeneous (between 1-4 hours, 162±102 minutes), and sample size and number of metabolites was smaller in SMI. However, good concordance in direction of changes and absolute pBI and uBI scores in multiple metabolites between SMI and PMI at time points 1, 2 and 4 hours were found. Examples of the top ranked metabolites include: malonic acid (SMI: −97 versus PMI: 84 [1 hour], −39 [2 hours], −76 [4 hours]), lactic acid (SMI: 37 versus PMI: −11, 30, 89) or glyceraldehyde (SMI: 35 versus PMI: 66, 84, 50). In the published study on PMI a focus lies on a group of metabolites that includes aconitic acid, hypoxanithine, trimethylamine N-Oxide (TMNO) and threonine that changed significantly in a sustained pattern at each of the 1, 2 or 4 hour time points after PMI, showing analogous behavior in direction and magnitude of changes in SMI as well. Therefore, pBI and uBI is a feasible model to qualitatively and quantitatively assess altered metabolic patterns in static and dynamic phenotypes, and confirm that metabolic biomarkers derived in the PMI model were similarly changed in the SMI samples, underscoring the effectiveness and the generalizibility of this approach.

It should be noted that because of the small sample size in both PMI and SMI cohorts, statistical reliability of pBI and uBI score thresholds is somewhat limited. Nonetheless, it could be seen that determined uBI score cut-offs in SMI data show concordance with pBI cut-offs estimated in PMI, and thus underscore the high degree of comparability and reliability between both scoring model variants.

To assess generalizability, the findings in PMI and SMI were compared, serving as another layer of validation that underscores the methodological and biological plausibility demonstrated within the present invention. A pool of metabolites associated with myocardial injury could be identified and verified, some of which by applying the scoring scheme at the level of weak, moderate or strong predictors, while other metabolites did not fulfil these criteria, but revealed high concordance in direction and magnitude of changes without achieving statistical significance. Nevertheless, differences in predictive value of metabolic biomarker candidates found in PMI versus SMI need to be further discussed from the perspective of heterogeneous sample collection relative to the exact onset of SMI, interindividual variability apparent in regular case-control versus longitudinal studies, and also due to differences in sub-cellular mechanisms in PMI compared with ‘normal’ patients with myocardial infarction.

While the invention has been illustrated and described in detail in the drawings and foregoing description, such illustration and description are to be considered illustrative or exemplary and not restrictive. It will be understood that changes and modifications may be made by those of ordinary skill within the scope and spirit of the following claims. In particular, the present invention covers further embodiments with any combination of features from different embodiments described above and below.

The invention also covers all further features shown in the Figures individually although they may not have been described in the afore or following description. Furthermore, in the claims the word “comprising” does not exclude other elements or steps, and the indefinite article “a” or “an” does not exclude a plurality. A single step may fulfil the functions of several features recited in the claims. The terms “essentially”, “about”, “approximately” and the like in connection with an attribute or a value particularly also define exactly the attribute or exactly the value, respectively. A computer program may be stored/distributed on a suitable medium, such as an optical storage medium or a solid-state medium supplied together with or as part of other hardware, but may also be distributed in other forms, such as via the Internet or other wired or wireless telecommunication systems. Any reference signs in the claims should not be construed as limiting the scope.

### REFERENCE LIST

Ackermann, B. L., Hale, J. E, Duffin, K. L. (2006) The role of mass spectrometry in biomarker discovery and measurement. Curr Drug Metab, 7, 525-39.
Baumgartner, C., Baumgartner, D. (2006) Biomarker discovery, disease classification, and similarity query processing on high-throughput ms/ms data of inborn errors of metabolism. J Biomol Screen, 11, 90-99.
Baumgartner, C., Graber, A. (2007) Data mining and knowledge discovery in metabolomics In: Masseglia F, Poncelet P, Teisseire M (eds.) Successes and new directions in data mining. IGI Global. Ch. 7, p. 141-166.
Bäckström, T., Goiny, M., Lockowandt, U., Liska, J., Franco-Cereceda, A. (2003) Cardiac outflow of amino acids and purines during myocardial ischemia and reperfusion. J Appl Physiol, 94, 1122-1128.
Collinson, P. O., Gaze, D. C. (2007) Biomarkers of cardiovascular damage and dysfunction—an overview. Heart Lung Circ, 16 Suppl 3, S71-82.
Cristianini, N., Shawe-Taylor, J. (2000) An introduction to support vector machines and other kernel-based learning methods. Cambridge University Press, Cambridge.
Dettmer, K., Aronov, P. A., Hammock, B. D. (2007) Mass spectrometry-based metabolomics. Mass Spectrom Rev, 26, 51-78.
Fawcett, T. (2006) An introduction to ROC analysis. Pattern Recogni Lett, 27, 861-874.
Gerszten, R. E., Wang, T. J. (2008) The search for new cardiovascular biomarkers. Nature, 451, 949-952.
Goldhaber, S. Z., Pohost, G. M., Kloner, R. A., Andrews, E., Newell, J. B., Ingwall, J. S. (1982) Inosine: a protective agent in an organ culture model of myocardial ischemia. Circ Res, 51, 181-188.
HaIl, M. A., Holmes, G. (2003) Benchmarking attribute selection techniques for discrete class data mining. IEEE T Knowl Data En, 15, 1437-1447.
Hosmer, D. W., Lemeshow, S, (2000) Applied logistic regression, 2^{nd} Edition. New York, N.Y., Wiley.
Howie-Esquivel, J., White, M. (2008) Biomarkers in acute cardiovascular disease. J Cardiovasc Nurs, 23, 124-131.
Jemal, M., Xia, Y. Q. (2006) LC-MS Development strategies for quantitative bioanalysis. Curr Drug Metab, 7, 491-502.
Kell, D. B. (2007) Metabolomic biomarkers: search, discovery and validation. Expert Rev Mol Diagn, 7, 329-133.
Larrañaga, P., Calvo, B., Santana, R., Bielza, C., Galdiano, J., Inza, I., Lozano, J. A., Armañanzas, R., Santafé, G., Pérez, A., Robles, V. (2006) Machine learning in bioinformatics. Brief Bioinform, 7, 86-112.
Lewis, G. D., Asnani, A., Gerszten, R. E. (2008a) Application of metabolomics to cardiovascular biomarker and pathway discovery. J Am Coll Cardiol, 52,117-23,
Lewis, G. D., Liu, E., Yang, E., Shim, X., Martinovic, M., Farrell, L., Asnani, A., Cyrille, M., Ramanathan, A., Shaham, O., Berriz, G., Lowry, P. A., Palacios, I., Tasan, M., Roth, F, P., Min, J., Baumgartner, C., Keshishian, H., Addona, T., Mootha, V. K., Rosenzweig, A., Carr, S. A., Fifer, M. A., Sabatine, M. S., Gerszten, R. E. (2008b) Metabolite profiling of blood from individuals undergoing planned myocardial infarction reveals early markers of myocardial injury. J Clin Invest, 118, 3503-3512.
Maisel, A. S., Peacock, W. F., McMullin, N., Jessie, R., Fonarow, G. C., Wynne, J., Mills, R. M. (2008) Timing of immunoreactive B-type natriuretic peptide levels and treatment delay in acute decompensated heart failure: an ADHERE (Acute Decompensated Heart Failure National Registry) analysis. J Am Coll Cardiol, 52, 534-540.
Mei, D. A., Gross, G. J., Nithipatikom, K. (1996) Simultaneous determination of adenosine, inosine, hypoxanthine, xanthine, and uric acid in microdialysis samples using microbore column high-performance liquid chromatography with a diode array detector. Ana Biochem, 238, 34-39.
Netzer, M., Millonig, G., Osl, M., Pfeifer, B., Praun, S., Villinger, J., Vogel, W., Baumgartner, C. (2009) A new ensemble-based algorithm for identifying breath gas marker candidates in liver disease using ion molecule reaction mass spectrometry (IMR-MS). Bioinformatics, 2009, 25, 941-947.
Osl, M., Dreiseitl, S., Pfeifer, B., Weinberger, K., Klocker, H., Bartsch, G., Schafer, G., Tilg, B., Graber, A., Baumgartner, C. (2008) A new rule-based data mining algorithm for identifying metabolic markers in prostate cancer using tandem mass spectrometry. Bioinformatics, 24, 2908-2914.
Sabatine, M. S., Liu, E., Morrow, D. A., Heller, E., McCarroll, R., Wiegand, R., Berriz, G. F., Roth, F. P., Gerszten, R. E. (2005) Metabolomic identification of novel biomarkers of myocardial ischemia. Circulation, 112, 3868-3875.
Saeys, Y., Inza, L, Larrañaga, P. (2007) A review of feature selection techniques in bioinformatics. Bioinformatics, 23, 2507-2517.
Saeys, Y., Abeel, T., Peer, Y. (2008) Robust feature selection using ensemble feature selection techniques. Proc. Eur. Conf. on Machine Learning and Knowledge Discovery in Databases. Part II. Lecture Notes in Artificial intelligence, 5212, 313-325.
Shulaev, V. (2006) Metabolomics technology and bioinformatics. Brief Bioinform, 7, 128-139.
Turgan, N., Boydak, B., Habif, S., Gülter, C., Senol, B., Mutaf, I., Ozmen, D., Bayindir, O. (1999) Urinary hypoxanthine and xanthine levels in acute coronary syndromes. Int J Clin Lab Res, 29, 162-165.
Witten, I. H., Frank, E. (2005) Data mining; Practical machine learning tools and techniques (2nd ed.). San Francisco: Morgan Kaufmann Publishers,
Zimmerman, J., Fromm, R., Meyer, D., Boudreaux, A., Wun, C. C., Smalling, R., Davis, B., Habib, G., Roberts, R. (1999) Diagnostic marker cooperative study for the diagnosis of myocardial infarction. Circulation, 99, 1671-1677.

1. A method for identification of biomarkers in a subject, the method comprising:
obtaining a sample from the subject;
measuring a plurality of biomolecules in the sample;
identifying the measured biomolecules;
estimating a discriminatory ability of each identified biomolecule by using a paired test hypothesis; and
integrating estimated discriminatory abilities of the identified biomolecules into a kinetic analysis.

2. The method according to claim 1, further comprising searching significant correlations and kinetic relations between early-appearing and later-appearing biomolecules in serial sampling studies based on the kinetic analysis.

3. The method according to claim 1, wherein estimating the discriminatory ability of each identified biomolecule comprises identification and categorization of biomarker candidates according to their predictive value.

4. The method according to claim 1, wherein the paired test hypothesis is evaluated according to the formula:
$$pBI = λ \cdot {DA}^{*} \cdot \sqrt{\frac{\vert {Δ}_{change} \vert}{\vert CV \vert}} \cdot sign ( {Δ}_{change} )$$
$$with$$
$${Δ}_{change} = \{ \begin{matrix}Δ & if Δ \geq 1 \\ - \frac{1}{Δ} & else\end{matrix}$$
wherein pBI is an paired Biomarker Identifier, λ is a scaling factor, DA* is the initial measure DA rescaled between 0 and 1, CV is a coefficient of variation, sign( ) determines the direction of change, Δ_{change} indicates changes in a biomolecule calculated as a relative increase or decrease from the levels of a reference cohort.

5. The method according to claim 1, wherein the discriminatory ability of each of the identified biomolecules is estimated by additionally using an unpaired test hypothesis.

6. The method according to claim 5, wherein the unpaired test hypothesis is evaluated according to the formula:
$$uBI = λ \cdot {TP}^{{2}^{*}} \cdot \sqrt{\vert {Δ}_{change} \vert \frac{{CV}_{ref}}{CV}} \cdot sign ( {Δ}_{change} )$$
$${Δ}_{change} = \{ \begin{matrix}Δ & if Δ \geq 1 \\ - \frac{1}{Δ} & else\end{matrix} \\ with \\ {Δ}_{ref} = \frac{\overline{x}}{\overline{x}}$$
wherein uBI is an unpaired Biomarker Identifier, λ is a scaling factor, TP²* is an initial measure defined as TP² rescaled between 0 and 1, Δ_{change} indicates changes in biomolecules calculated as a relative increase or decrease from the levels of a reference group, CV is a coefficient of variation, CV_{ref} is a reference coefficient of variation, sign( ) determines the direction of change, x̅ is the mean value from the levels of a biomolecule in a cohort, x̅_{ref} is the mean value from the levels of a biomolecule in a reference cohort.

7. The method according to claim 1, wherein the biomolecules are selected from a group consisting of nucleotides, amino acids, organic acids, sugars, lipids, acyl-carnitines, peptides or proteins.

8. The method according to claim 1, wherein the sample comprises a body fluid or tissue.

9. The method according to claim 8, wherein the body fluid is blood and urine, blood or urine.

10. A method of using biomarkers identified in a subject, the method comprising:
obtaining a sample from the subject;
measuring a plurality of biomolecules in the sample;
identifying the measured biomolecules;
estimating a discriminatory ability of each identified biomolecule by using a paired test hypothesis;
integrating estimated discriminatory abilities of the identified biomolecules into a kinetic analysis; and
predicting risk of future life threatening events in disease, prognosis or diagnosis based on the kinetic analysis.

11. The method according to claim 10, wherein disease is selected from the group consisting of metabolic diseases including cardiovascular disease or cancer.

12. A method for monitoring progress or treatment of a disease, the method comprising:
(a) providing numerical scores for biomarkers based on the discriminatory ability by using the method according to claim 1, wherein the scores are predetermined to be relevant to the disease;
(b) repeating (a) after a period of time during which subjects receive treatment for the disease, to obtain post-treatment scores;
(c) comparing the post-treatment scores from (b) with the scores from (a) versus scores for subjects not suffering under the disease, and
(d) classifying the treatment as being effective if scores from (b) are closer to the scores from (a) than to the scores for normal subjects.

13. The method according to claim 12, wherein the provision of numerical scores for biomarkers is carried out in at least one of a disease cohort versus a healthy cohort study and longitudinal biomarker cohort study.

14. The method according to claim 12, wherein the period of time is within a range of minutes, hours, days or months.

15. The method according to claim 12, wherein disease is selected from the group consisting of metabolic diseases including cardiovascular disease or cancer.
